(19) United States
(12) Patent Application Publication
(10) Pub. No.: US 2003/0104446 A1
(43) Pub. Date: Jun. 5, 2003

(54) Method for detecting and characterising activity of proteins involved in lesion and dna repair

(76) Inventor: Sylvie Sauvaigo, Vizille (FR)

Correspondence Address: BURNS DOANE SWECKER & MATHIS L L P, POST OFFICE BOX 1404, ALEXANDRIA, VA, 22313-1404, US

(21) Appl. No.: 10/258,858

(22) PCT Filed: May 23, 2001
(86) PCT No.: PCT/FR01/01605

(30) Foreign Application Priority Data
May 24, 2000 (FR) 00/06624

(51) Int. Cl. C12Q 1/68

(52) U.S. Cl. 435/6

(57) ABSTRACT

The invention relates to a method for detecting and characterising the activity of protein(s) involved in the repair of DNA, comprising the following steps:

a) fix a known damaged DNA O comprising a lesion (7) onto a solid support (1)

b) subject this damaged DNA to the action of a repair composition that may contain at least one protein contributing to the repair of this damaged DNA, and

c) determine the activity of this protein for the repair, by measuring the variation of the signal emitted by a marker (5) that is fixed onto or is eliminated from the support in step b).

### TECHNICAL FIELD

The purpose of this invention is a method for detecting and characterising the activity of proteins involved in the repair of DNA lesions.

It is particularly applicable to the study of enzymes and protein factors involved in the repair of DNA and to the study of the genotoxicity of chemical substances or physical agents in living systems.

### STATE OF PRIOR ART

Lesions or damages can form in cellular DNA by exposure to various chemical or physical agents, particularly after irradiation (ionising and solar radiation), after induction by an oxidising stress, or after exposure to some genotoxic or cytotoxic agents. Damaged nucleosides may also accumulate naturally in the genome during the cellular aging process.

There are several types of lesions or damage to nucleic acids, that are frequently characteristic of the agent that induced them. These types of damage include single and double-strand breaks, abasic sites and modifications to nucleic bases.

Different repair mechanisms are involved depending on the nature of the lesion, and they may even come into competition. A distinction can be made between three main repair mechanisms; repair of mismatches, repair by excision of nucleotides (REN) and repair by excision of bases (REB). Furthermore, the corresponding recombination that is a cellular mechanism involved during meiosis, enables the repair of double-strand DNA breaks.

These mechanisms involve different repair proteins. Thus, the repair mechanism by excision of nucleotides (REN) involves several steps, as follows:

1) recognition of the damage by a protein complex,

2) incision of the strand containing the lesion, on each side of the lesion,

3) excision of an oligonucleotidic fragment containing the lesion, and

4) synthesis of the new good DNA strand with a final ligation.

Many proteins and enzymes are involved in this mechanism, which mainly repairs light induced lesions and large volume damages caused by chemical treatments.

The base excision repair (REB) mechanism uses a family of enzymes called glycosylases. These are sometimes specific to the substrate to be excised, but usually recognise modification categories. Some glycosylases also have endonuclease activities. These enzymes repair oxidised, fragmented or alkylised bases. This system also includes abasic sites, some mismatches, and some single strand breaks.

Six glycosylases have been identified in humans, (Wilson III, D. M and Thompson, L. H., 1997, Proc. Natl. Acad. Sci., 94, pages 12754-12757 [9]), which are useful mainly to repair deaminated, oxidised, alkylised bases, or for the correction of some mismatches. The action of glycosylases results in the formation of an abasic site recognised by a specific endonuclease that incises the phosphodiester bond at 5′ from the eliminated base. A polymerase replaces the eliminated nucleotide and a ligase closes the nucleotidic chain. Note that some glycosylases have an associated lyase activity that incises the phosphodiester bond at 3′ from the lesion and causes the formation of a 5′-phosphate end. There is an alternative to this REB method during which a small damaged fragment of DNA is eliminated and then replaced. This could be relevant for some single strand DNA breaks. Specific enzymes coded by identified genes are thus associated with excision of the defined lesions.

Methods designed to evaluate cellular repair activities are complicated and difficult to implement. They frequently require the use of chemically modified plasmids. The repair capacity of cellular extracts is measured in vitro by repairing these modifications correlated to the incorporation of marked nucleotides, during the resynthesis of excised strands. The method was initially developed by Wood et al., Cell, 53, 1988, pages 97-106 [1]. Superwound plasmids are modified by C type UV radiation, or acetyl-aminofluorene , or cisdichlorodiamnineplatine. Plasmids are then incubated in a medium containing cellular extracts, the four deoxinucleosidetriphosphates, one of which is marked in the α position; by a ³²p, ATP and an ATP regeneration system. Lesions can be eliminated during incubation by nucleotide excision or base excision systems. The DNA resynthesis rate is determined after migration of DNA on agarose gel and after counting the radioactivity of the searched strip. The importance and the specificity of the repair are related to the quality of the extracts and the plasmid, the lesions created and the reaction parameters, as described by Sallès et al., Biochimie, 77, 1995, pages 796-802 [2]. In particular, an initial plasmidic preparation is necessary to eliminate plasmids containing breaks before treatment or bacterial chromosome fragments that would supply polymerisation initiation sites.

Another disadvantage of this technique is related to the lesions created. The treatments used do not induce a single defect. For example, most damage generated by UVC radiation are the cyclobutane type of pyrimidine dimers. But other lesions are formed such as photoproducts, chain breaks, cytosine hydrates, etc. Thus, particular monitoring of the repair of each type of damage is not easy using these substrates.

Document FR-A-2 731 711 [3] describes a method for detecting DNA lesions consisting of fixing the DNA on a solid phase and then creating lesions on this DNA by means of a product producing lesions and using cellular extracts containing repair factors and a marker. The purpose of this system is to be able to measure genotoxic effects of some chemical substances. DNA in plasmid form is firstly fixed on a solid phase (microplates well) and the plasmids are then chemically modified. The repair reactions are made in the presence of modified triphosphate nucleosides that enable non radioactive detection of neo-synthesised strands. This system is incapable of incorporating specific modifications in a targeted manner at the required location. It is a system capable of detecting a global effect of a DNA modifying agent without identifying lesions recognised by repair systems. Furthermore, the purpose of this method is not to detect and quantify the activity of proteins involved in the DNA repair, but rather to identify the presence of lesions on the treated DNA.

Page et al., Biochemistry, 29, 1990, pages 1016-1024 [4] and Huang et al., Proc. Natl. Acad. Sci., 91, 1994, pages 12213-12217, [5], use a different test to measure the damage excision capacities using cellular extracts from various origins. Specific lesions are created or incorporated into short synthesis oligonucleotides. These are then bonded to each other by ligases to give longer double-strand fragments (150 to 180 pairs of bases). The incorporation of ³²p at different positions on the fragments provide a means of localising break sites created by the repair enzymes. The analysis is made by autoradiography after electrophoresis on acrylamide gel. This test is applicable to REB and REN type repairs.

There are disadvantages with all the methods described above. In most cases, radioactive marking and long separations by electrophoresis are necessary to determine the excision of damages. The use of plasmids is difficult to implement since precautions have to be taken to guarantee their purity and to minimise the background noise. Furthermore, as already mentioned, it is impossible to induce a single type of damages by one stress and several lesions are thus simultaneously present in the DNA.

Methods are also known for studying specific features and damage excision mechanisms using repair enzymes on 15 to 50 long base synthesis oligonucleotides containing well defined lesions, as described by Romieu et al., J. Org. Chem., 63, 1998, pages 5245-5249 [6] and D'Ham et al., Biochemistry, 38, 1999, pages 3335-3344 [7].

In most cases, the oligonucleotide containing the chemical modification(s) to be studied is radioactively marked at one of its ends. The action of the enzyme (excision kinetics, determination of affinity constants, break or no break of the oligonucleotide fragment, action on a single or double strand) is analysed on acrylamide gel after electrophoresis.

Another technique avoids the use of radioactivity but requires a large investment; this is the analysis of digestion by mass spectrometry (MADI-TOF). This analysis is interesting because it enables a study of the excision mechanism. On the other hand, it is absolutely not suitable for a routine analysis of an enzymatic activity.

Excision of modified bases may also be followed by gaseous chromatography coupled with mass spectrometry. This was demonstrated starting from the excision of 5hydroxy-5,6-dihydrothymine and 5,6-dihydrothymine by endonuclease III on DNA irradiated by γ radiation. There are several disadvantages with this precise technique; it requires a large investment in analytic material, it is not very sensitive and requires a large amount of raw material, and it is not suitable for routine analyses.

### PRESENTATION OF THE INVENTION

The purpose of the invention is a method for detecting and characterising proteins involved in the repair of DNA damages that is easier to implement, adaptable to different repair modes, that can be used easily and quickly in a routine, that does not use radioactivity.

According to the invention, the method for detecting and characterising one or several activities of protein(s) involved in repair of DNA comprises the following steps:

a) fix at least one damaged DNA comprising at least one known lesion, onto a solid support,

b) apply the action of a repair composition that may or may not comprise at least one protein contributing to the repair of this damaged DNA, and,

c) determine the activity of this (these) protein(s) for the repair by measuring the variation of the signal emitted by a marker that fixes onto or is eliminated from the support in step b).

In this process, the damaged DNA in which at least one lesion is known, is fixed onto a solid support such as a biochip and it is then subjected to the action of a protein such as a repair enzyme, or a composition that may contain this protein, and the repair is then monitored by using a marker that may initially be fixed to the damaged DNA or added into it during the repair process.

Thus, the process can be used to characterise proteins involved in the repair of the DNA. It can also be used to demonstrate non-functionality of some proteins with regard to known lesions, or to detect the lack of DNA repair proteins in compositions that normally should contain them.

It is useful to obtain these results for diagnostic applications. In some diseases, repair genes are muted and their enzymatic activity or the associated protein are not functional or are only partially functional (for example xeroderma pigmentosum).

In this process, the solid support comprises at least one fixation site defined by a fragment of damaged DNA and advantageously comprises several fixation sites capable of immobilising different previously chosen damaged DNA fragments.

The proteins for which the activity is to be measured are proteins involved in the repair process that generally comprise recognition of the damage, an incision of the DNA chain, excision of the damage or of fragments of nucleic acids, an in situ synthesis of the DNA, and ligation of the neoformed strand.

For example, the proteins involved in this process may be chosen from among:

proteins involved in the reconnaissance of lesions such as XPA, the TFIIH transcription factor and its constituent polypeptides, XPC, XPF, XPG and their associated proteins (ERCC family, etc.)), HSSB, etc. (see Sancar, A. (1995) Annu. Rev. Genetics, 29, pages 69-105, [10]),

proteins involved in the excision of lesions such as glycosylases,

proteins involved in the resynthesis of nucleotide(s) of the excised strand such as polymerases, and

proteins involved in the ligation of neoformed strands such as ligases.

According to a first embodiment of the process according to the invention, the marker is present on the damaged DNA fixed on the support and it is eliminated by the action of the protein in step b).

For example, this embodiment may be used to determine the activity of incision or excision enzymes for the damaged DNA lesions. In this case, the marker may be present on one end of the damaged DNA. Thus, an incision or excision eliminates the damaged DNA fragment carrying the marker and causes loss of the signal on the support at the location at which the damaged DNA is fixed.

The first embodiment may also be used with a specific marker for lesions of the damaged DNA such as an antibody, which reveals these lesions before step b) After the protein has acted and the lesions have been repaired, this marker can no longer be fixed and a loss of signal of the marker representative of the activity of the repair protein may be observed.

Thus, the ratio of the signal emitted by specific antibodies before the DNA was repaired to the signal emitted by the antibodies after the protein reaction, for example an enzymatic reaction, is correlated to the DNA repair ratio. In this case, the damage repair is evaluated by the disappearance of a signal specific to the damage.

According to a second embodiment of the process according to the invention, the marker is present in the repair composition and is added into the DNA fixed on the support in step b).

In this case, it is possible for example to add modified nucleotides incorporated by polymerases and that can be used for marking of the neoformed strand, into the composition. These modified nucleotides may carry a biotin, a hapten, a fluorescent compound or any other molecule compatible with marking of nucleic acids. These markers introduced during the resynthesis step may be subsequently detected and the corresponding signal correlated to a repair ratio.

Markers that can be used to determine the activity of proteins involved in the repair of DNA may be different types, provided that they emit a signal that can be detected or that can be revealed by emitting a detectable signal.

Several markers or development methods may be used simultaneously or in sequence so as to demonstrate state changes that occurred on damaged DNA fragments, by protein activities related to the repair.

In particular, the marker may be an affinity molecule, a fluorescent compound, an antibody, a hapten or a biotin.

Preferably, according to the invention, the marker or marker developer may consist of fluorescent compounds with direct fluorescence or indirect fluorescence. For example, these molecules may be avidine revealed by steptavidine-phycoerithrine, europium cryptates, fluorescent compounds such as fluoresceines, rhodamine, etc.

Energy transfer properties between fluorescent molecules may also be used to quantify an enzymatic activity involved in the repair on a substrate oligonucleotide. If the oligonucleotide comprising the base(s) with lesions also carries a fluorescent molecule authorising transfer of energy with another fluorescent molecule located either on the same oligonucleotide or on an oligonucleotide in contact with the first, an emission of a signal after excitation proves that the lesion is present on the support. The incision of the oligonucleotide comprising the lesion(s) by a repair enzyme will cause elimination of the incised fragment and loss of the fluorescent signal, and the energy transfer cannot take place. This signal change can be measured and correlated to a specific digestion rate.

According to the invention, a damaged DNA may be fixed on the solid support using conventional processes.

Thus, a damaged DNA can be synthesised directly on the solid support by means of automatic synthesisers that can for example incorporate modified bases.

The damaged DNA may also be fixed on the solid support by any method compatible with maintaining the integrity of the damaged DNA fragment, for example by hybridising with another DNA fragment immobilised on the support, by immobilisation by an affinity molecule, or by direct fixation on the chip by deposition or by any other means.

Preferably, a biochip will be used as a solid support on which DNA fragments comprising different or identical but perfectly identified lesions, or comprising multiple lesions not perfectly identified, are fixed at determined locations.

The immobilized damaged DNA fragments may be short oligonucleotides (15-100 bases long, preferably 15 to 50 bases long) or longer fragments (100-20 000 bases) Different methods exist to incorporate oligonucleotides comprising lesions in long DNA fragments: Chain Polymerisation Reaction (PCR), ligation. Immobilised DNA fragments may be in single or double strand form. The modified DNA may be attached directly or through an intermediate molecule (biotin, antigen, nucleic acid type, etc.).

Long DNA fragments may also be damaged by any physical or chemical treatment inducing lesions. For example, they may be irradiated by radioactive, solar or ultraviolet radiation. They may be damaged by light sensitising, by chemical agents, by carcinogens. In this case, the lesions are induced statistically along the nucleotidic chain. The quality, specificity and quantity of induced lesions depends on the choice of the chemical or physical agent that causes the lesions. Thus, the category of lesions to be introduced in a DNA fragment can be targeted.

For example, the support may be a biochip containing DNA fragments, on which the damaged DNA is fixed by hybridising by adding an oligonucleotide comprising a complementary part of the damaged DNA and a complementary part of one of the DNA fragments of the biochip.

The invention benefits from the advantages of the chemical synthesis of oligonucleotides and therefore the fact that it is possible to choose the exact sequences containing and surrounding the damage.

The same support may be functionalised by nucleotic fragments with identical or different sequences. The damages may be located at different locations in the sequences, that may or may not be defined.

The nature of fragments fixed on the support (size, number and location of each damage, etc.) can be varied and depends on the nature of the required information.

DNA fragments comprising lesions or complementary fragments of fragments comprising lesions may also include markers that are useful for detecting DNA transformations related to an enzymatic repair activity. These markers may be affinity molecules, fluorescent compounds or any specific detection system related to a specific type of chip.

When the DNA fragments are immobilised on the chip (reference chip), a development is made in order to characterise the states in which the fragments are found. The different pins of the chip are thus characterised by the signals that they emit. They are used as a reference signal.

The reference chip, or a chip comprising damaged DNA fragments identical to those on the reference chip, called the test chip is then incubated in the presence of solutions that might contain enzymatic repair activities. Modified DNA fragments fixed on the chip may be transformed by the enzymatic activities present.

A second reading of the test chip or the reference chip is made under appropriate conditions and after the necessary development steps. The pins of the chip may thus emit signals different from the signals in the first reading, meaning that one of several enzymatic reactions have taken place on the chip.

Note that the measurement of the transformation of DNAs damaged by enzymes related to the repair systems may be monitored by making a continuous record of the signals emitted by the different pins on the chip, if the markers used are compatible with such a detection system.

The process according to the invention may be used for the study of enzymes of purified protein factors and for the study of activities present in cellular extracts.

It may also be used to study the specific features of different DNA lesions for some repair enzymes, particularly for glycosylases. In the same field, repair kinetics for these lesions may be monitored as a function of different factors. These factors may be related to the nucleotidic sequence (for example, the effect of surrounding bases on the repair) or inhibitors, or on the other hand to repair activity simulators.

It is interesting to be able to monitor the repair of an identified lesion for the assay of enzymatic activities involved in the reconnaissance and excision steps for a particular lesion. For example, it is known that specific glycosylases exist for excision of a particular lesion. Therefore a deficiency of a particular enzyme will prevent a particular lesion from being repaired.

Miniaturisation of the support is a means of improving the sensitivity of the system, particularly when cellular extracts are used. The invention facilitates detection of the different proteins related to repair systems starting from culture cells or biopsies. Furthermore, if a biochip comprising several damaged DNA sequences with different known lesions is used, the capacities for repair of each damage for a limited quantity of the same cellular extract can be detected. This is very important, particularly when making a diagnosis of some pathologies and genetic diseases.

In particular, the invention has a large number of advantages.

It enables miniaturisation of systems for the assay of enzymatic activities, and more particularly activities related to repair. The result is an improvement in sensitivity. This improvement is very important, because cellular extracts are difficult to obtain, particularly when they originate from biological samples and not from cell lines.

It can be used to obtain precise information about the function, induction and repression of different steps related to the repair of damages to DNA in procaryote or eucaryote systems.

It thus makes it easier to access biological assays of enzymatic activities in man. In particular, it enables detection of enzymatic deficiencies that may be found in pathologies involving radiosensitivity or photosensitivity such as Xeroderma pigmentosum, Cockaine's disease. It also makes it easier to study the effects of aging on enzymatic repair activities.

The invention is also a means of simultaneously studying the behaviour of enzymes with regard to different lesions incorporated into nucleotidic fragments immobilised on the same miniaturised support. This facilitates a comparison between results and makes them more reliable and reproducible. Another result is an important time saving.

For the first time, the invention provides a global approach towards the evaluation of the function of repair systems with access to several lesions and several mechanisms or repair steps simultaneously.

A direct analysis of signals emitted by different pins of the chip after action by the enzymatic systems to be studied, can give direct information about the steps in the various repair systems and make it possible to reach a conclusion about how they function.

The invention enables the assay of a particular protein involved in the repair of a targeted lesion, for example the assay of a glycosylase involved in excision of oxidised bases. In this case, the lesion known as being the preferred substrate for the targeted glycosylase will be included in a synthesis ogligonucleotide and fixed on the chip at a specific location. Therefore, it can be seen that several oligonucleotides comprising different targeted lesions can be fixed at different locations on the chip, to obtain an evaluation of the function of different glycosylases involved in the REB system. But, the invention can also enable the assay of proteins involved in steps common to the repair of all lesions such as polymerases and ligases. In this case, DNA fragments comprising less precisely identified lesions can be used.

The invention can also be characterised by the fact that the choice of the different substrates fixed at different pins of the chip can be used to target the repair system in which we are interested. The fixation of short oligonucleotides (shorter than 25 bases) comprising lesions is a means of targeting proteins in the REB system. The fixation of longer fragments (longer than 50 bases) comprising lesions is a means of targeting proteins in the REB and the REN system at the same time. A precise selection of the fixed damages then makes it possible to target the repair system to be studied.

Thus, the process according to the invention can be used for studying the specific nature and function of proteins involved in the repair with regard to known DNA lesions, to monitor the kinetics of repairs to DNA lesions by proteins, to evaluate the genotoxic effect of substances or physical agents inhibiting or stimulating the synthesis of proteins involved in the repair of DNA, or for the diagnostic of deficiencies of repair proteins related to diseases.

Other characteristics and advantages of the invention will become clearer after reading the following examples, which are obviously given for illustrative purposes and in no way restrictive, with reference to the appended drawings.

### BRIEF DESCRIPTION OF THE DRAWINGS

FIG. 1 diagrammatically shows the state of a system in the first step of the process according to the invention.

FIG. 2 diagrammatically shows the state of the system in FIG. 1 after step b) in the process according to the invention.

### DETAILED DESCRIPTION OF EMBODIMENTS

### EXAMPLE 1

The first embodiment of the process according to the invention is used in this example to determine a glycosylase type repair activity on a damaged oligonucleotide comprising an 8-oxo-7,8 dihydro-2′-deoxiguanosine as a lesion.

A MICAM pb4 type biochip is used, obtained as described by Livache et al., Biosens. Bioelectron, 13, 1998, pages 629 and 634 [8], comprising four pins functionalised by four synthesis oligonucleotides with different sequences (H, I, J, K). The H sequence is as follows:

H sequence: 5′ TTTTT CCA CAC GGT AGG TAT CAG TC.

The functionalised part of the biochip is incubated in the presence of a hybrid formed from the damaged oligonucleotide (O) comprising an 8-oxo-7,8-dihydro-2′-deoxiguanosine and an oligonucleotide and a (cOcH) comprising a complementary part of the O oligonucleotide (cO) and a complementary part of the H oligonucleotide fixed on the biochip (cH). The O oligonucleotide comprises a biotin at its end 3′.

O: 5′GAA CTA GTG XAT CCC CCG GGC TGC-Biotin 3′

(where X is 8-oxo-7,8 dihydro-2′-deoxiguanosine).

cOcH: 5′ GCA GCC CGG GGG ATC CAC TAG TTC GAC TGA CTA CCG TGT GG;

The O-cOcH hybrid is obtained by incubation of 10 pmoles of cOcH and 12 pmoles of 0 in 200 μl of PBS buffer solution containing 0.2 M of NaCl, for 60 minutes at 37° C. 20 μl of this solution is drawn off and is added to the functionalised part of the chip that forms a dish. After one hour of incubation at 37° C. in a moist environment, the chip is soaked in a washing buffer (PBS/0.2 M NaCl/tween 20 0.05%).

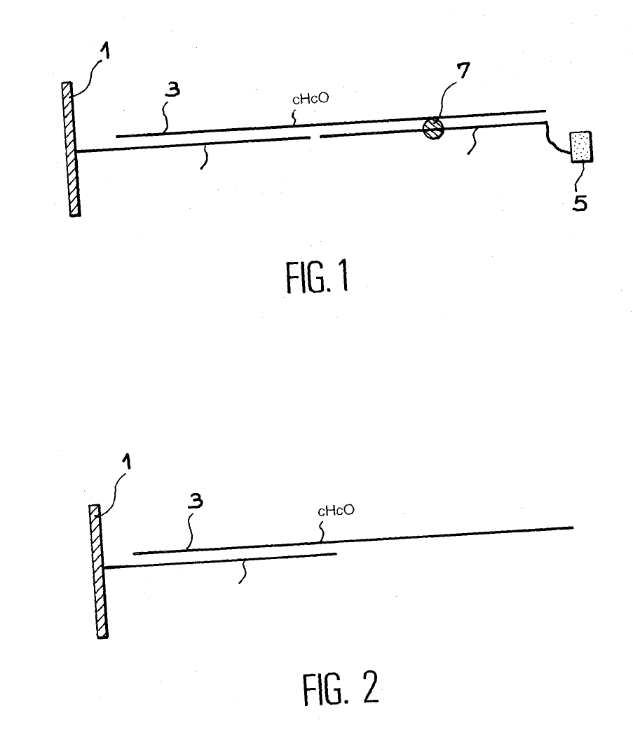

FIG. 1 shows the system obtained by fixation of the O oligonucleotide on pin 1 of the biochip comprising the H oligonucleotide. This H oligonucleotide is hybridised with the cH sequence of the oligonucleotide 3 that also comprises a sequence hybridised with the damaged O oligonucleotide carrying a biotin 5 at its end 3′ and a lesion 7.

The O oligonucleotide comprising the lesion and fixed to the biochip by hybridisation is developed after incubation for 10 minutes at ambient temperature with 20 μl of PBS/NaCl buffer containing 0.5% of bovine albumin serum and 1 μl of streptavidine-phycoerythrine R (0.5 mg/ml; Jackson ImmunoResearch).

After washing in PBS/NaCl/tween, the biochip is observed under a fluorescence microscope and the signal is analysed using the Image Pro Plus software.

The signal is integrated in pixels. The functionalised pins emit a weak fluorescent signal even if there is no hybridisation. Non-functionalised pins are black.

An intense saturating fluorescent signal is observed on the functionalised pin by the H oligonucleotide at 250 pixels. For comparison, the pin functionalised by the I sequence has a maximum fluorescence of about 110 pixels. The signal to noise (H/I) ratio is 2.27.

Therefore, hybridisation of the oligonucleotide comprising a DNA lesion is specific. Furthermore, it can be seen that a fragment of nucleic acid comprising defined damage on a predefined site on a biochip can be fixed.

The biochip is washed with 0.1 N NaOh for 10 minutes and is then rinsed with H₂O for 10 minutes, which has the effect of eliminating the O and cOcH oligonucleotides from the biochip. The microscope is used to check that all signals have disappeared.

The chip is hybridised again with the O-cOcH hybrid under the conditions defined previously. After washing with PBS/NaCl/tween, the chip is balanced for 10 minutes with a 0.1 M KCl , Tris buffer, and then 20 μl of this buffer containing 0.5 μg of Fapy DNA Glycosylase (S. Boiteux, CEA Fontenay-aux-Roses, France) is added. The chip is incubated at 37° C. for 30 minutes in a moist atmosphere.

After washing with PBS/NaCl/tween, the development step is carried out in the same way as before using streptavidine-phycoerythrine R. The signal is recorded after washing with PBS/tween. The intense fluorescence on the H pin has disappeared. The signal to noise ratio (H/I) is 1.04.

FIG. 2 shows the state of the system after reaction with the glycosylase type enzyme.

This figure shows that the O oligonucleotide has been eliminated from the biochip. The Fapy DNA glycosylase enzyme cut the O oligonucleotide at 8-oxo-7,8-dihydro-deoxiguanosine. The short fragments of DNA thus generated form instable hybrids under the conditions used and are eliminated from the biochip. This causes disappearance of the fluorescence signal due to development of the biotin.

This example thus shows that a DNA repair activity can be detected on a microsupport carrying a fragment of DNA with a lesion of a DNA base.

### EXAMPLE 2

This example illustrates the detection of a glycosylase type activity in a cellular lysate using the first embodiment of the process according to the invention.

In this example, the 0 oligonucleotide used in example 1, comprising 8-oxo-7,8-dihydro-deoxiguanosine is hybridised as in example 1 on the biochip pb4, which corresponds to the system shown in FIG. 1.

A total cellular lysate is prepared starting from the Hela cells in culture.

The cells are trypsinised and then washed in a PBS buffer. The base containing about 15×10⁶ cells is dissolved in 1 ml of lyse buffer (Tris-HCl 10 mM pH 7.5, MgCl₂ 10 mM, KCl 10 mM, EDTA 1 mM, containing 1 pellet for 10 ml of antiproteases “Complete, Mini” (Boehringer Mannheim) and 5 μl of Phenylmethylsulphonyl Fluoride (PMSF, Sigma, 17.4 mg/ml in isopropanol). The cells are ground and then the lysate is centrifuged in a Beckman ultracentrifuge at 4° C. for 50 minutes at 65 000 rpm. The floating material is recovered and 200 μl of glycerol and 20 μl of dithiotreitol 0.1 M are then added. The lysates are aliquoted and stored at −80°

The biochip is then incubated with 20 μl of cellular extract for 45 minutes at 37° C. Development is performed as in example 1.

The signal from the H pin is recorded and is 180 pixels.

The experiment is repeated with denatured lysate (heating to 100° C. for 10 minutes). The signal from the H pin is saturating at 250 pixels. Therefore, there is a signal loss following incubation of the biochip functionalised by the duplex containing the damage with the untreated cellular lysate. This signal loss is about 30%, corresponding to a partial cut-off of the O oligonucleotide modified by enzymes contained in the lysate.

These experiments are confirmed by analysis on polyacrylamide gel after radioactive marking of the O oligonucleotide.

### EXAMPLE 3

This example illustrates detection of the excision/resynthesis repair activity of a total cellular lysate, using a modified DNA fragment fixed on a microsupport.

The second embodiment of the process according to the invention is used in this case.

A DNA fragment consisting of 5000 pairs of bases is prepared by PCR amplification from the lambda phage using the “Expand™ Long template PCR system” kit by Roche. One of the amplification primers comprises a sequence of 15 bases (J₁₅) at its end 5′ separated by an amino synthon from the sequence hybridising on the phage. This sequence remains a single strand after PCR amplification. This PCR fragment is purified by the micro-spin S300 column (Amersham-Pharmacia). The DNA strand is irradiated by C type ultra-violet rays for 3 minutes (γₘₐₓ 254 nm, 0.8 J/cm²).

The irradiated DNA is then hybridised by means of a oligonucleotide cI₁₅cJ₁₅ 30 bases long, complementary firstly for the I₁₅ sequence and secondly for the J₁₅ sequence, on a pb2 biochip (MICAM) comprising four different oligonucleotide sequences on four different pins (S1, S2, S3, I₁₅), including the sequence called I₁₅.

I₁₅: 5′ TTTTT ATC CGT TCT ACA GCC

Hybridisation takes place during 1 hour at 37° C. in 20 μl of PBS/NaCl buffer containing about 0.1 pmole of the PCR amplification product.

The chip is then incubated in the presence of total cellular extracts in a buffer adapted as described above.

Excision-resynthesis experiment on the functionalised chip:

The protocol is adapted from Robins et al., the EMBO Journal, col. 10, No. 12, pages 3913-3921, 1991 [10]. A solution is prepared containing 25 μl of cellular lysate of Hela cells, 10 μl of 5×reactional buffer (Hepes KOH 225 mM pH 7.8, KCl, 350 mM, MgCl₂ 37.5 mM, DTT 4.5 mM, EDTA 2 mM, BSA 0.09 mg/ml, glycerol 17%), ATP 2 mM, dGTP 5 μm, DATP S μM, dCTP 5 μM, dTTP 1 μM, Biotine-16-2′-deoxiuridine-5′-triphospate 4 μM, phosphocreatine 200 mM, creatine phosphokinase type I 12.5 μg in a total volume of 50 μm. 25 μl of this solution is deposited on the biochip that is incubated for 2h30 at 30° C. in a moist environment.

After rinsing with PBS/NaCl/tween, the development is made with streptavidine-phycoerythrine.

The chip is then analysed with a microscope.

Strong fluorescence is observed (saturating signal) at the pin functionalised by the I₁₅ oligonucleotide. The signal to noise ratio (I₁₅/S3) is 2.2.

A streptavidine-phycoerythrine development carried out before repair of the modified DNA fragment by the enzymes contained in the lysate gives a signal to noise ratio (I₁₅/S3) equal to 1.

The experiment is repeated with the unmodified fragment obtained by PCR reaction. No signal can be seen on the chip after the excision/resynthesis reaction carried out under the same conditions as before (signal/noise 1).

This example shows that the enzymatic activities involved in the repair of modified bases of DNA can be detected on a chip functionalised by a fragment of DNA comprising modified bases.

### EXAMPLE 4

This example illustrates the use of specific antibodies for demonstrating lesions composed of cyclobutane type pyrimidine dimers.

The same biochip will be used as in example 3, in which DNA irradiated using the same oligonucleotide cI₁₅cJ₁₅ under the same conditions as in example 3 is fixed.

After washing with PBS/NaCl/tween, the cyclobutane type pyrimidine dimers formed by the UVB irradiation are developed.

The chip is incubated with 20 μl of PBS/NaCl of buffer containing 1 μl of anti-dimer antibody (500 μg/ml; Kamiya Biomedicam Company, Seattle, USA), for 1 hour at 37° C.

After washing with PBS/NaCl/tween the chip is incubated in the presence of 20 μl of PBS/NaCl containing 1 μl of anti-mouse goat antibody coupled with the Cy™3 marker (1.4 mg/ml; Jackson ImmunoResearch Laboratories, Inc.) for 1 hour at 37° C.

After washing with PBS/NaCl/tween, the signal is recorded on the chip. The signal to noise ratio (I₁₅/S3) is 1.33.

Thus, the use of the DNA anti-damage antibodies specifically detects damages and can therefore be used to monitor their elimination by repair enzymes.

### REFERENCES MENTIONED

[1]: Wood et al., Cell, 53, 1988, pages 97-106.

[2]: Sallès et al., in Biochimie, 77, 1995, pages 796-802.

[3] FR-A-2 731 711.

[4]: Page et al., Biochemistry, 29, 1990, pages 1016-1024.

[5]: Huang et al., Proc. Natl. Acad. Sci., 91, 1994, pages 12213-12217.

[6]: Romieu et al., J. Org. Chem., 63, 1998, pages 5245-5249.

[7]: D'Ham et al., Biochemistry, 38, 1999, pages 3335-3344.

[8]: Livache et al., Biosens. Bioelectron, 13, 1998, pages 629-634

[9]: Wilson III, D. M. and Thompson, L. H., 1997, proc. Natl. Acad. Sci., 94, pages 12754-12757.

[10]: Sancar, A. (1995) Annu. Rev. Genetics, 29, pages 69-105.

1. Method for detecting and characterising one or several activities of protein(s) involved in the repair of DNA, comprising the following steps:
a) fixing onto a solid support at several defined locations several damaged DNAs composed of oligonucleotides having identical sequences having different known lesions and/or of oligonucleotides having different sequences and identical or different know lesions,
b) subjecting this damaged DNA to the action of a repair composition that may or may not contain at least one protein involved in the repairing of this damaged DNA, and,
c) determining the activity of this (these) protein(s) of the repairing by measuring the variation of the signal emitted by a marker that is fixed onto or is eliminated from the support in step b).

2. Process according to claim 1, in which the protein is chosen among the proteins involved in the reconnaissance of DNA lesions, the proteins involved in excision of DNA lesions, the proteins involved in the resynthesis of the nucleotide(s) of the excised strand, and proteins involved in ligation of neoformed strands.

3. Process according to claim 1, in which the marker is present in the damaged DNA fixed onto the support and is eliminated by action of the protein in step b).

4. Process according to claim 3, in which the marker is fixed to one end of the damaged DNA, the protein is an incision enzyme or excision enzyme of the lesions of the damaged DNA, and the incision or excision causes elimination of a fragment of the damaged DNA carrying the marker.

5. Process according to claim 3, in which the marker is a specific marker of damaged DNA lesions that could reveal these lesions before step b) and the lesion is eliminated during the repair such that the marker can no longer reveal the lesion.

6. Process according to claim 1, in which the marker is present in the repair composition and is introduced into the DNA fixed on the support in step b).

7. Process according to claim 6, in which the repair composition comprises a nucleotide modified by the marker.

8. Process according to claim 1, in which the repair composition is a cellular lysate or a purified repair enzyme.

9. Process according to claim 1, in which the marker is an affinity molecule, a fluorescent compound, an antibody, a hapten or a biotin.

10. Process according to claim 1, in which the support is a biochip comprising DNA fragments on which the damaged DNA is fixed by hybridising using an oligonucleotide comprising a complementary part of the damaged DNA and a complementary part of one of the DNA fragments of the biochip.

11. Process according to claim 1, in which the damaged DNA is a short oligonucleotide of from 15 to 100 bases long, preferably of from 15 to 50 bases long, comprising lesions incorporated into the oligonucleotide during its chemical synthesis.

12. Process according to claim 1, in which the damaged DNA is a polynucleotide of from 100 to 20 000 bases long.

13. Process according to claim 1, in which the damaged DNA is in the form of a single or double strand.

14. Biochip on which several damaged DNAs are fixed composed of oligonucleotides having an identical or different sequence, with known different lesions and/or oligonucleotides identical or different lesions, at several defined locations.

15. Use of the process according to any one of claims 1 to 13, for the study of the specific nature and functionality of proteins involved in the repairing with regard to known DNA lesions.

16. Use of the process according to any one of claims 1 to 13, to monitor the kinetics for the repairing of DNA lesions by proteins.

17. Use of the process according to any one of claims 1 to 13, to evaluate the genotoxic effect of substances or physical agents inhibiting or stimulating the synthesis of proteins involved in the repairing of the DNA.

18. Use of the process according to any one of claims 1 to 13, for the diagnostic of deficiencies of repairing proteins related to diseases.
